(19) United States
(12) Patent Application Publication
(10) Pub. No.: US 2011/0172726 A1
(43) Pub. Date: Jul. 14, 2011

(54) CRANIAL ELECTROSTIMULATION METHOD AND EQUIPMENT

(75) Inventors: Martin Reinier Snyman, Western Cape Province (ZA); Jaen Frederik Labuschagne, Western Cape Province (ZA)

(73) Assignee: TO BE FIRST AG, Zug (CH)

(21) Appl. No.: 13/119,169

(22) PCT Filed: Sep. 17, 2009
(86) PCT No.: PCT/IB09/06868

§ 371 Date: Mar. 15, 2011

(30) Foreign Application Priority Data
Sep. 18, 2008 (GB) 0817089.6

(51) Int. Cl. A61N 1/36

(52) U.S. Cl. 607/3

(57) ABSTRACT

A method and equipment for conducting cranial electrostimulation on a patient are provided. Suitable electrodes are attached to the head of the patient cause electrostimulation currents to be created in response to pulses applied to the electrodes. Music is simultaneously audible to the patient. The pulses, or sequences of pulses, are generated at a frequency that is varied according to the rhythm, beat of music that is audible to the patient. The equipment comprises a cranial electrostimulation pulse generator and associated electrodes for applying pulses to the head of a patient, and a sound signal generator with at least one associated loudspeaker that is typically a pair of earphones, for generating audible sound. A controller controls the frequency at which pulses or sequences of pulses are generated by the cranial electrostimulation pulse generator such that the frequency is varied according to the rhythm, beat or tempo of the music.

### FIELD OF THE INVENTION

This invention relates to a cranial electrostimulation method such as that often referred to as electrotherapy and to equipment for use in carrying out the method. More especially, but not necessarily exclusively, the invention relates to an electrostimulation method in the form of what is known as cranial electro-biologic stimulation that is typically applied between electrodes clipped to the earlobes of a patient receiving treatment.

It is to be understood that the term “patient” as used in this specification is intended to be interpreted broadly as being any person being subjected to electrostimulation irrespective of whether the underlying reason for applying such electrostimulation has curative intentions or not. In this regard it is noted that electrostimulation is often used with the intention of simply improving a patient's quality of life such as by improving the patient's mood, emotions, attitude or cognitive capabilities.

### BACKGROUND TO THE INVENTION

Cranial electro-biologic stimulation (herein referred to as CES) applies gentle micro-current pulses to the brain (in the hypothalamic area) using a pair of ear-clip electrodes that attach to the earlobes. It is widely accepted that CES stimulates the brain to manufacture neurotransmitters, like endorphins, which improve moods, emotions and cognitive capabilities. Cranial electrostimulation has also been proposed for treatment following a stroke, brain trauma, certain heart disorders, high blood pressure, jet leg, motion sickness and dementia.

The signals apparently normalise the electrical output of the brain. CES has thus been used/tested to treat substance dependence, depression and anxiety. It has been noted in at least some instances that CES has equal or greater efficacy for the treatment of depression when compared to antidepressant medications, with fewer side effects.

The mechanism by which CES produces its effects is not yet fully understood. It is postulated that the stimulation of brain tissue causes increased amounts of neurotransmitters to be released, specifically serotonin, beta endorphin, and noradrenaline. It is believed that these neurotransmitters in turn permit a return to normal biochemical homeostasis of the limbic system of the brain that may have been imbalanced by a stress-related condition.

Treatments that have typically been used range from 10 to 30 minutes in duration although they may extend up to 1½ hours depending on the electrical current configuration. Typically the currents employed would be applied in pulse form with a pulse width in the range of from about 1 to about 500 milliseconds (ms) at a frequency of from about 0.1 Hertz (Hz) to about 500 Hz with the current being typically less than 1 milliampere (mA) and more typically about 300 to 700 microamperes (μA), although currents of up to about 2.5 milliampere (mA) have also been used.

The beneficial effects of music on the human body have long been appreciated and employed in the treatment, in particular, of psychological disturbances.

### OBJECT OF THE INVENTION

It is an object of this invention to provide a cranial electrostimulation method and equipment aimed at having an enhanced overall effect on a patient.

### SUMMARY OF THE INVENTION

In accordance with one aspect of this invention there is provided a method of conducting cranial electrostimulation on a patient comprising attaching suitable electrodes to the head of the patient and causing electrostimulation currents to be created in response to pulses applied to the electrodes and simultaneously causing music to be audible to the patient, the method being characterised in that the pulses, or sequences of pulses, are generated at a frequency that is varied according to the rhythm, beat or tempo of music that is audible to the patient.

Further features of this aspect of the invention provide for the pulse width to also be dependent on the rhythm, beat or tempo of music that is audible to the patient such that the average current remains generally constant over the range of different frequencies; for the electrical pulses to fall within the general ambit considered as cranial electro-biologic stimulation; for the electrodes to be contacted with the ears of the patient, in particular with any one or more of the pinna and earlobe of the ear, the entrance to the auditory canal, and within the auditory canal; for the music to be audible by way of earphones typically positioned at the entrance to the auditory canal or within the auditory canal in which instance the electrodes may optionally be carried by the earphones so as to contact a patient's skin in the operative position; and for the music to be either instrumental or vocal, or both.

The beat could be a binaural beat.

In accordance with a second aspect of the invention there is provided equipment for the implementation of a method as defined above, said equipment comprising a cranial electrostimulation pulse generator and associated electrodes for applying pulses generated by the pulse generator to the head of a patient, and a sound signal generator and at least one associated loudspeaker for converting output from the sound signal generator into audible sound, the equipment being characterised in that it includes a controller for controlling the frequency at which pulses or sequences of pulses are generated by the cranial electrostimulation pulse generator such that the frequency is varied according to the rhythm, beat or tempo of music audible by way of the said at least one loudspeaker.

Further features of this aspect of the invention provide for the at least one loudspeaker to be a pair of earphones; for the cranial electrostimulation pulse generator and sound signal generator to be built into a single unit; for such single unit to be a portable unit that is preferably battery powered; for the equipment to include a data base containing a selection of different musical numbers that can be selected by a user; and for the equipment to have manually operable controls and an interface display screen for facilitating the selection of a treatment procedure involving one or more musical numbers or a predetermined sequence of musical numbers selected to provide a particular treatment.

Still further features of the second aspect of the invention correspond to those of the first aspect of the invention.

In order that the above and other features of the invention may be more fully understood different embodiments thereof will be described below with reference to the accompanying drawings.

### BRIEF DESCRIPTION OF THE DRAWINGS

In the drawings:—

FIG. 1 is a block diagram of one embodiment of method and equipment for use in exercising this invention;

FIG. 2 is a perspective illustration of a portable version of equipment operating on the basis of the block diagram of FIG. 1;

FIG. 3 is a perspective illustration of one form of earphone for use with the equipment illustrated in FIG. 2;

FIG. 4 is an exploded illustration thereof;

FIG. 5 illustrates in FIG. 5a an earphone about to be installed in an ear; in FIG. 5b the earphone installed; and in FIG. 5c the area of contact of the electrode with the ear;

FIG. 6 is an elevation from the outside of a multi-electrode earphone having electrodes arranged to contact the ear externally on or adjacent the pinna;

FIG. 7 illustrates in FIG. 7a the inside of a multielectrode earphone illustrated in FIG. 6; in FIG. 7b the multielectrode earphone open; and in FIG. 7c felt covers for the electrodes;

FIG. 8 illustrates in FIG. 8a the multielectrode earphone open and about to be installed on an ear; in FIG. 8b the earphone installed on the ear, and in FIG. 8c zones in which the various electorates contact the ear;

FIG. 9 illustrates an alternative form of multi-electrode earphone for introduction into the entrance of the auditory canal;

FIG. 10 is a graphic illustration of a sequence of different coupled pairs of electrodes in a multielectrode arrangement;

FIG. 11 is a block diagram of one arrangement for selecting channels and polarity in a multielectrode arrangement; and,

FIG. 12 is a block diagram of an alternative arrangement for selecting channels and polarity in a multielectrode arrangement.

### DETAILED DESCRIPTION WITH REFERENCE TO THE DRAWINGS

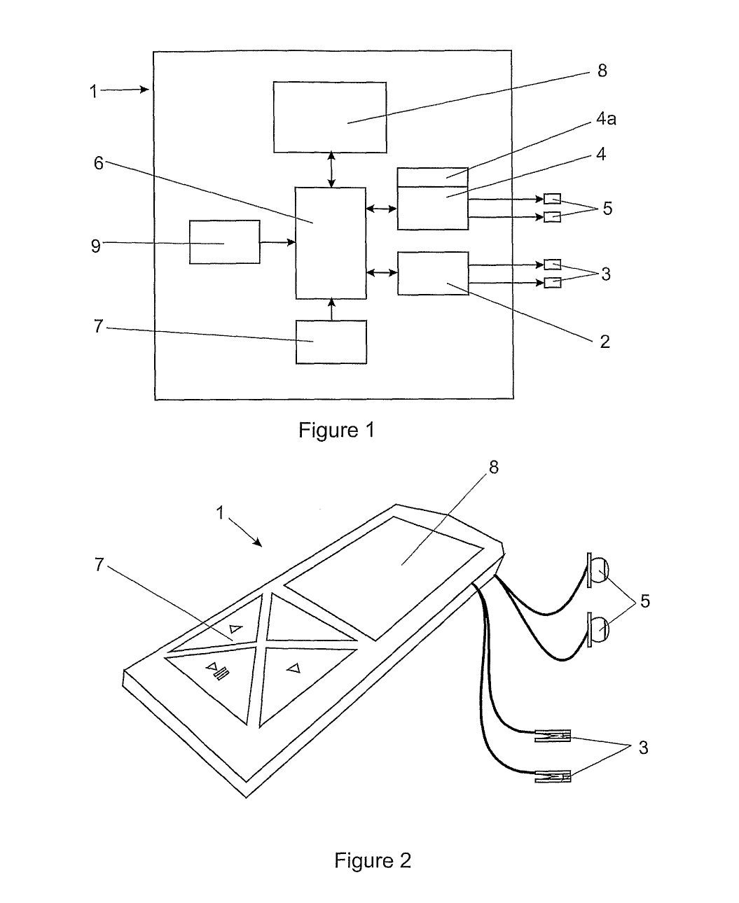

In the embodiment of the invention illustrated in FIGS. 1 and 2, the method of the invention is implemented by way of equipment that in one instance is a portable battery powered unit (1) that may take the shape and configuration of a pocket sized therapy unit, as illustrated in FIG. 2.

The therapy unit is configured to carry out CES on a patient by the application of appropriate electrical pulses to the ears simultaneously with the playing of music that is audible to the patient. The equipment thus comprises a cranial electrostimulation pulse generator (2) and associated electrodes (3) for applying pulses generated by the pulse generator to the ears of a patient, and a sound signal generator (4) and a pair of earphones (5) for converting output from the sound signal generator into audible sound. The sound signal generator may have associated with it a data base containing a selection of different musical numbers that can be selected by a user or by selecting a predetermined treatment programme or session. The data base is indicated by numeral (4a) in FIG. 1.

The electrodes for attachment to the ears of a patient may be of the conventional type that clip onto the ear lobes in which instance the earphones could be of substantially conventional type commonly employed for listening to music using any one of many different digital media players. Indeed the sound signal generator could be, or be based on, the electronic circuitry of such a digital media player.

However, as will become more apparent from what follows, it is preferred that the electrodes and earphones be combined into combination units that either fit into the entrance to the auditory canal; within the auditory canal itself; or are held adjacent the entrance to the auditory canal but spaced somewhat therefrom.

Reverting to the unit itself, the outputs from the electrostimulation pulse generator and sound signal generator are controlled by a controller in the form of a microprocessor (6) that may be user operated by a user button interface (7). The unit has a display screen (8) for assisting in programme selection in very much a conventional manner. Obviously the display screen could be replaced by any other suitable indicator means such as an array of light emitting diodes or the like. A battery charger circuit (9) is also provided.

The above arrangement is such that the electrostimulation pulse generator produces electrical pulses having a frequency and pulse width selected to provide a required microcurrent across the head, in use, such microcurrent typically being of the order of 500 to 750 μA and, in any event, less than 1 mA. The frequency could range from 0.5 Hz to about 3 Hz and the pulse width could range from about 125 ms at a frequency of 0.5 Hz to about 75 ms at a frequency of 3 Hz. The microcurrent across the head is, in any event, generally maintained at a substantially constant average current

As provided by this invention, the pulses (or possibly sequences of pulses in the event that predetermined sequences of optionally different pulses are employed) are generated at a frequency that is dependant on the tempo of music that is being played at any one time and that is audible to the patient by way of the earphones. Of course, in order to achieve a substantially constant average current, the pulse width of the pulses will also vary with variation in frequency. The microprocessor monitors the tempo of the music and controls the generation of the electrical pulses on the basis thereof.

Whilst not in anyway being limiting to the scope of the invention it is proposed that the following could operate effectively:—

At an audio tempo of 30 beats per minute a frequency of 0.5 Hz could be employed with a pulse width of 125 ms;

at an audio tempo of 60 beats per minute a frequency of 1.0 Hz could be employed with a pulse width of 115 ms;
at an audio tempo of 90 beats per minute a frequency of 1.5 Hz could be employed with a pulse width of 105 ms;
at an audio tempo of 120 beats per minute a frequency of 2.0 Hz could be employed with a pulse width of 95 ms;
at an audio tempo of 150 beats per minute a frequency of 2.5 Hz could be employed with a pulse width of 85 ms; and,
at an audio tempo of 180 beats per minute a frequency of 3.0 Hz could be employed with a pulse width of 75 ms.

The microprocessor is also programmed to provide treatment programmes or sessions of a predetermined duration, as is well known in the art, and the duration would generally be somewhere between 10 minutes and 30 minutes but may extend up to an hour or even an hour and a half. Information as to any particular treatment session to be chosen, musical numbers and the like can be displayed on the screen (8) provided on the therapy unit.

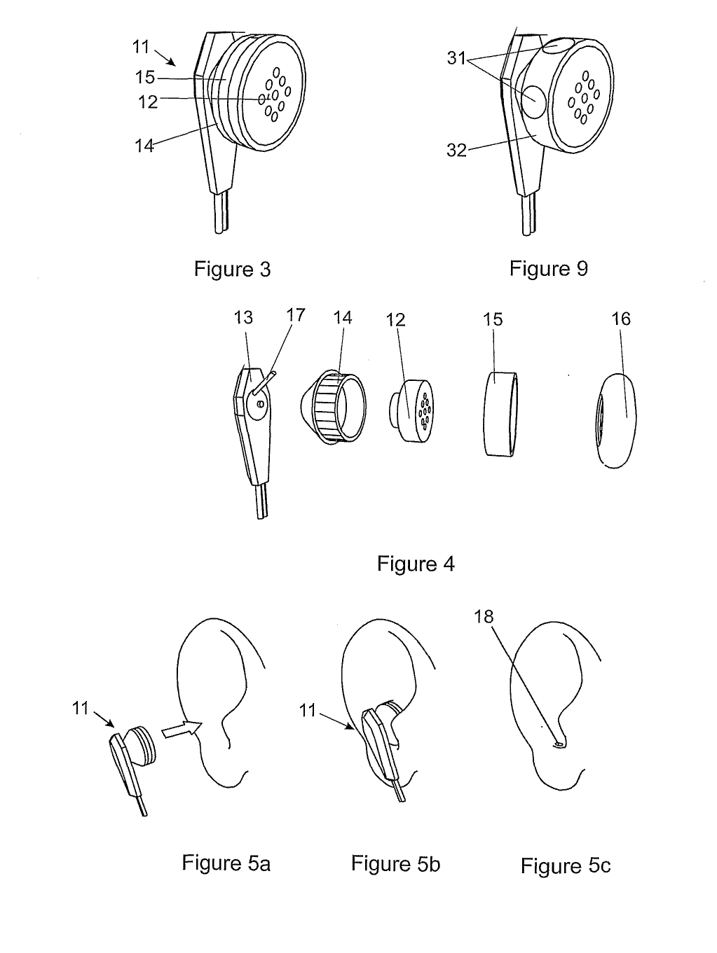

Reverting now to the nature of the earphones, and with particular reference to FIGS. 3 to 5 of the accompanying drawings, each earphone unit (11) could comprise a standard audio earphone (12) mounted on a base (13) and surrounded by an insulating ring (14). A ring-shaped electrode (15) surrounds the insulating ring and is covered by an electrically conductive rubber sheath (16). The electrode is, of course, electrically connected to the electrostimulation pulse generator (2) by way of a suitable conductor (17).

As shown in FIG. 5, the earphone is adapted to be received in the entrance to the auditory canal so that the ring makes connection with the skin, for example in the region indicated by numeral (18) in FIG. 5c. Of course the earphone could equally well assumed the form of one that is accommodated within the auditory canal, in use, such earphones often been referred to as canal phones or earbuds.

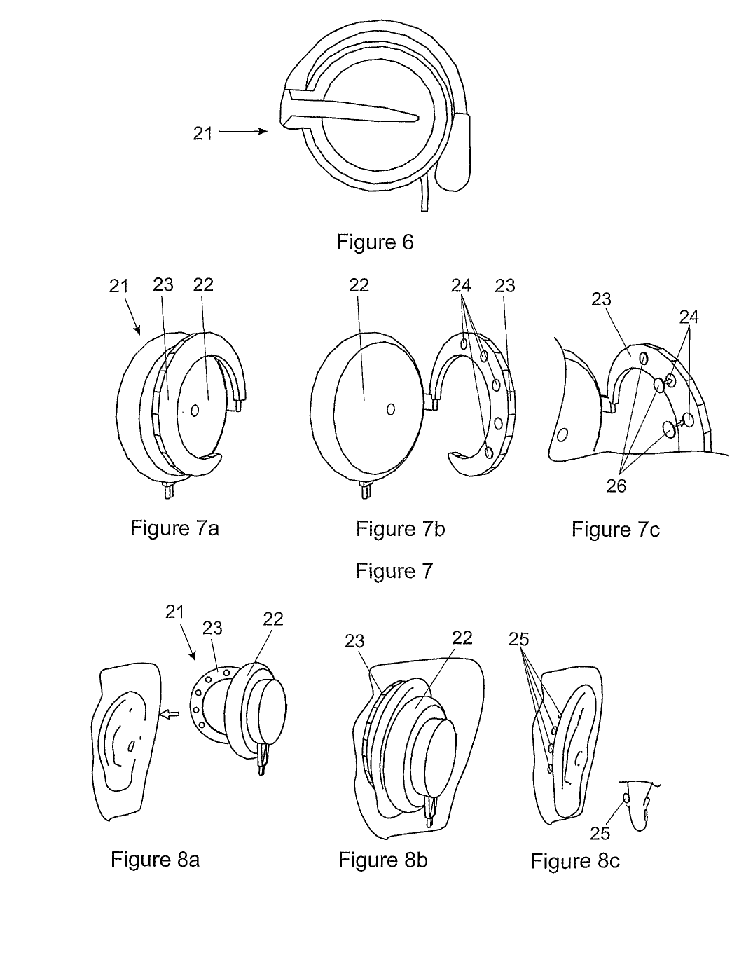

It is envisaged that the invention can also be applied by way of multielectrode units that are illustrated in FIGS. 6 to 8 of the accompanying drawings. In this instance an earphone unit (21) has an earphone (22) of the type shaped to be held against the ear a short distance outwards from the entrance to the auditory canal and an arcuate electrode carrier (23) that can be swung outwards away from the sound emitting face of the earphone. The electrode carrier can thus be swung inwards to engage the rear face of the pinna of a person's ear to hold the earphone unit in position. In the operative position multiple electrodes (24) in the face of the arcuate electrode carrier contact the rear of the pinna of the ear at arcuately spaced positions that are indicated by numeral (25) in FIG. 8c. Each individual electrode is preferably covered by an electrically conductive felt patch (26) or the like, as shown in FIG. 7c.

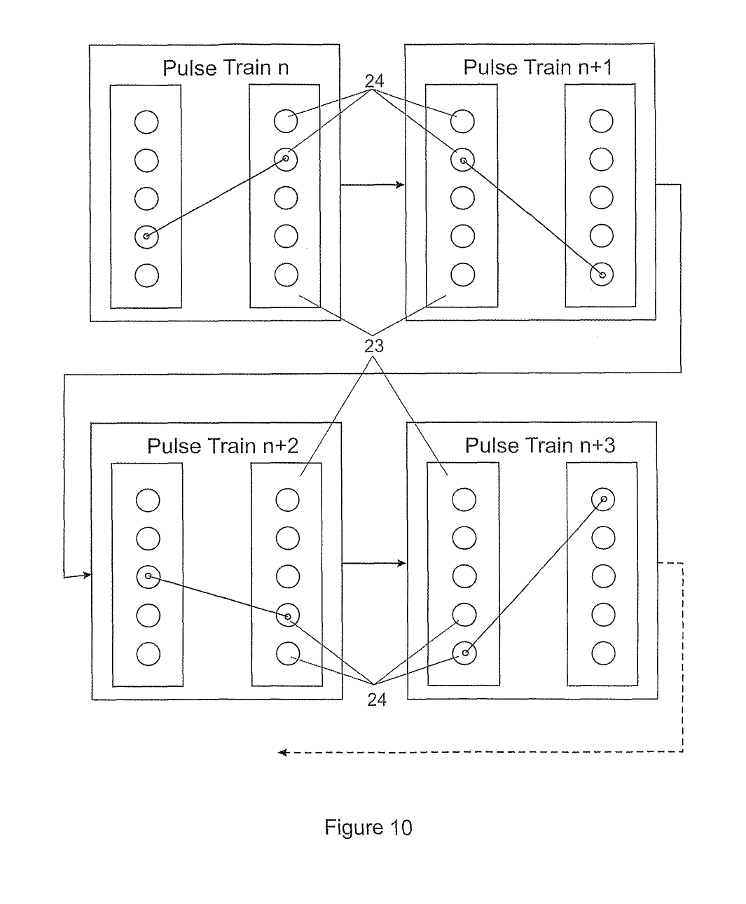

In this variation, the arrangement is such that current is only induced between one electrode of each earphone unit at any one time and different pairs of cooperating electrodes are selected sequentially or randomly via the microprocessor. FIG. 10 indicates some sequential connections between single electrodes of each multiplicity thereof simply by way of example.

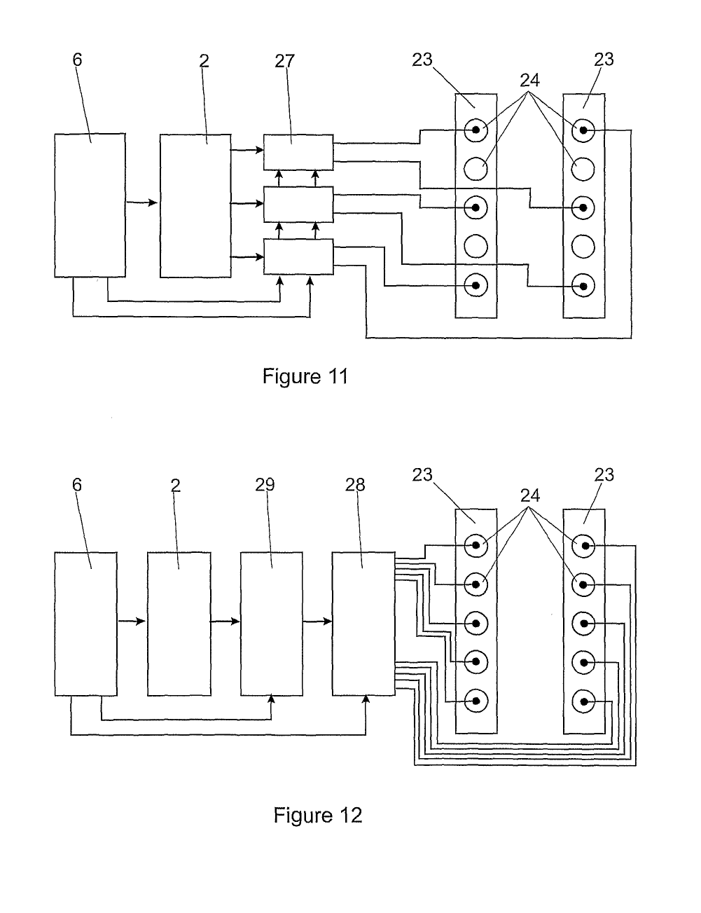

In one type of arrangement that is illustrated in FIG. 11, each electrode pair to be used in a circuit is allocated a channel (27) and the microprocessor can randomly, or in a programmed manner, activate a channel and select a direction of the current by setting the polarity of the electrodes of a pair. This arrangement may be rather hardware intensive in the event that many channels are to be used.

Another type of arrangement is illustrated in FIG. 12 in which a multiplexing type of arrangement is illustrated and the microprocessor selects pairs of electrodes of each of the multiplicities randomly, or in a programmed manner, and each circuit is closed utilizing an analogue switch unit (28). In this arrangement the polarity is controlled by the microprocessor by way of a direction control unit (29).

It is envisaged that the use of multielectrode earphone units will most likely provide different electrical paths through the cranial region with a likely enhancement of the therapeutic value being obtained consequent on a treatment.

FIG. 9 illustrates an alternative multielectrode earphone unit in which the multiple electrodes (31) are spaced apart on a cylindrical insulating surface (32) of the type of earphone adapted to be received in the entrance to the auditory canal or in the auditory canal itself.

In each instance in which multielectrode earphone units are employed, the pulses of electrical energy are varied according to the tempo of the music being played.

It will be understood that numerous variations may be made to the various aspects of the invention described above without departing from the scope hereof. In particular, numerous different arrangements of electrode are possible within the scope of the invention, those identified above simply being examples.

Also, as regards the equipment generally, the electrostimulation pulse generator and sound signal generator do not need to be embodied in a single unit, especially when used in a treatment centre or studio, depending on the objectives to be achieved and the duty that the equipment is required to perform.

1. A method of conducting cranial electrostimulation on a patient comprising attaching suitable electrodes to the head of the patient and causing electrostimulation currents to be created in response to pulses applied to the electrodes and simultaneously causing music to be audible to the patient, the method being characterised in that the pulses, or sequences of pulses, are generated at a frequency that is varied according to the rhythm, beat or tempo of music that is audible to the patient.

2. A method as claimed in claim 1 in which the pulse width is also dependent on the rhythm, beat or tempo of music that is audible to the patient such that the average current remains generally constant over the range of different frequencies.

3. A method as claimed in claim 1 in which the electrical pulses fall within the general ambit considered as cranial electro-biologic stimulation.

4. A method as claimed in claim 1 in which the electrodes are contacted with the ears of the patient.

5. A method as claimed in claim 4 in which the electrodes are contacted with any one or more of the pinna, earlobe, entrance to the auditory canal and the auditory canal itself of the patient.

6. A method as claimed in claim 1 in which the music is audible by way of earphones positioned adjacent, at or in the auditory canal in which instance the electrodes are carried by the earphones so as to contact a patient's skin in the operative position.

7. Equipment for the implementation of a method as defined in claim 1, said equipment comprising a cranial electrostimulation pulse generator and associated electrodes for applying pulses generated by the pulse generator to the head of a patient, and a sound signal generator and at least one associated loudspeaker for converting output from the sound signal generator into audible sound, the equipment being characterised in that it includes a controller for controlling the frequency at which pulses or sequences of pulses are generated by the cranial electrostimulation pulse generator such that the frequency is varied according to the rhythm, beat or tempo of music audible by way of the said at least one loudspeaker.

8. Equipment as claimed in claim 7 in which the at least one loudspeaker is a pair of earphones.

9. Equipment as claimed in claim 8 in which the electrodes are carried by the earphones so as to contact a patient's skin in the operative position.

10. Equipment as claimed in claim 7 in which the cranial electrostimulation pulse generator and sound signal generator are built into a single unit.

11. Equipment as claimed in claim 10 in which the single unit is a portable unit that is optionally battery powered.

12. Equipment as claimed in claim 7 in which the equipment includes a data base containing a selection of different musical numbers that can be selected by a user.

13. Equipment as claimed in claim 7 in which the equipment has manually operable controls and an interface display screen for facilitating the selection of a treatment procedure involving one or more musical numbers or a predetermined sequence of musical numbers selected to provide a particular treatment.
